(19) United States
(12) Patent Application Publication
(10) Pub. No.: US 2015/0131781 A1
(43) Pub. Date: May 14, 2015

(54) MOVABLE DIAPHRAGM UNIT, X-ray GENERATING APPARATUS AND X-RAY IMAGING SYSTEM

(71) Applicant: CANON KABUSHIKI KAISHA, Tokyo (JP)

(72) Inventors: Yasuo Ohashi, Kawasaki-shi (JP); Kazuyuki Ueda, Tokyo (JP)

(21) Appl. No.: 14/535,145

(22) Filed: Nov. 6, 2014

(30) Foreign Application Priority Data
Nov. 11, 2013 (JP) 2013-232708

(51) Int. Cl. A61B 6/00

(52) U.S. Cl. CPC A61B 6/4035; A61B 6/542
USPC 378/62; 378/121; 378/150

(57) ABSTRACT

A movable diaphragm unit is configured such that, when a restricting blade is moved away from a normal line extending from a center of a focal spot of X-ray to a first virtual plane including a detector plane, a moving distance of a crossing line of a second virtual plane including an end surface of the restricting blade and a third virtual plane including the focal spot with respect to a center of the focal spot becomes shorter than a moving distance with respect to the normal line of the end surface.

### BACKGROUND

1. Field of the Invention

The present disclosure relates to an X-ray imaging system applicable to medical equipment, a nondestructive inspection apparatus and the like. More particularly, the present invention relates to a medical X-ray imaging system capable of reducing unnecessary X-ray exposure of a subject and an X-ray imaging system using the X-ray imaging system.

2. Description of the Related Art

An X-ray imaging system includes a radiation generating apparatus and an X-ray detecting device. The radiation generating apparatus typically includes an X-ray generating tube in a container and includes a movable diaphragm unit on a front side of an X-ray emission window provided in this container. The movable diaphragm unit has a function to adjust an X-ray field by shielding, using a restricting blade, a portion unnecessary for imaging among the X-ray emitted from the X-ray generating tube and to reduce exposure of the subject to X-ray.

As a movable diaphragm unit, Japanese Patent Laid-Open No. 2009-90035 discloses a movable diaphragm unit capable of adjusting an opening diameter by moving a plate-shaped leaf (i.e., a restricting blade) on a curved surface. In such a movable diaphragm unit, the restricting blade has a predetermined thickness necessary to attenuate the X-ray in order to prevent X-ray from leaking in an unnecessary direction.

In the X-ray generating apparatus disclosed in Japanese Patent Laid-Open No. 2009-90035, the restricting blade rotates on a curved surface of which radius of curvature is a distance significantly shorter than a distance between the restricting blade and the focal spot of X-ray. Therefore, there has been a problem that, when the restricting blade opens widely, an effective thickness of the restricting blade at a corner on an opening side thereof is reduced and X-ray partially passes through the restricting blade, whereby unnecessary exposure of a subject increases.

Therefore, it has been necessary to provide an X-ray generating apparatus capable of optimizing an angle of the restricting blade with respect to a direction in which the X-ray is emitted and reducing unnecessary exposure of a subject. The present invention provides an X-ray imaging system capable of reducing unnecessary exposure and an X-ray imaging system using the X-ray imaging system.

### SUMMARY

An X-ray imaging system, including: a radiation generating apparatus which includes an X-ray generating unit configured to emit X-ray from a target upon irradiation of the target with an electron beam, and a movable diaphragm unit which includes a restricting blade configured to restrict a passage range of the X-ray and a moving mechanism of the restricting blade; and

an X-ray detecting device configured to detect, on a detector plane, X-ray emitted from the radiation generating apparatus and has passed through an object,

wherein the restricting blade has a predetermined thickness defined by a front surface on a target side and back surface of the opposite side of the front surface, and includes an end surface which communicates with the front surface and the back surface on a side on which the X-ray passes,

the moving mechanism moves the restricting blade in a direction in which the restricting blade crosses the end surface,

wherein, when a width of an eclipsed penumbra formed by X-ray emitted from a focal spot defined by an electron beam flux applied to the target, is in contact with the end surface, and arrives at a first virtual plane which includes the detector plane is denoted by h1 and a width of an attenuated penumbra formed by X-ray emitted from the focal spot, enters from the end surface, passes through the restricting blade, and arrives at the first virtual plane is denoted by h2, a relationship h2<h1 is satisfied,

a second virtual plane which is in contact with the end surface crosses a third virtual plane which includes the focal spot,

when the restricting blade is moved away from a normal line extending from a center of the focal spot to the first virtual plane, a moving distance of a crossing line of the second virtual plane and a third virtual plane with respect to a center of the focal spot is shorter than a moving distance of the end surface with respect to the normal line.

An X-ray imaging system including: the X-ray imaging system according to claim 1; and a control device configured to control the radiation generating apparatus and the X-ray detecting device in the X-ray imaging system in a cooperated manner.

Further features of the present invention will become apparent from the following description of exemplary embodiments with reference to the attached drawings.

### BRIEF DESCRIPTION OF THE DRAWINGS

FIG. 1 is a cross-sectional view schematically illustrating a configuration of an embodiment of an X-ray imaging system.

FIG. 2 is an enlarged view of a movable diaphragm unit in FIG. 1.

FIG. 3 is a cross-sectional view schematically illustrating a configuration of a movable diaphragm unit of an embodiment of an X-ray imaging system.

FIG. 4A is a cross-sectional view and FIG. 4B is a perspective view schematically illustrating a moving mechanism of the movable diaphragm unit of FIG. 3.

FIG. 5A and FIG. 5B are cross-sectional views schematically illustrating a configuration of a movable diaphragm unit of another embodiment of the X-ray imaging system.

FIG. 6 is a block diagram schematically illustrating a configuration of an embodiment of an X-ray imaging system.

FIG. 7 is a cross-sectional view schematically illustrating an exemplary configuration of a radiation generating apparatus.

FIG. 8A is a schematic sectional view and FIGS. 8B to 8D are schematic plan views of a restricting blade illustrating a configuration of an application of a movable diaphragm unit.

FIG. 9 is a perspective view illustrating an application of a shape of a restricting blade illustrated in FIG. 8.

FIGS. 10A to 10C are block diagrams illustrating an application of a movable diaphragm unit according to the present invention and FIG. 10C is a block diagram illustrating a state in which the radiation generating apparatus and the X-ray detecting device are connected by a support unit.

### DESCRIPTION OF THE EMBODIMENTS

In an X-ray imaging system, a main factor that defines a resolution of an X-ray imaging image is a focal diameter in a target which is an X-ray source. The focal spot of X-ray emitted by the X-ray imaging system according to the present invention is substantially defined by a focal spot of an electron beam flux applied to a target from an electron emission source. Hereafter, in this specification, the focal spot of the electron beam defined by the electron beam flux on the target will be referred to as a focal spot.

From a viewpoint of improving the resolution of the X-ray imaging image described above, it is desirable for the focal diameter to be small as much as possible. From a viewpoint of heat resistance of a material which constitutes the target and output intensity of X-ray, lower and upper limits of an anode current density and a focal diameter which flow in the target are defined, respectively. Typically, the lower limit of the focal diameter is set to be several tens of micrometers from a viewpoint of heat-resistant of the target, and the upper limit of the focal diameter is set to be several mm from a viewpoint of resolution.

In a movable diaphragm unit as described in Japanese Patent Laid-Open No. 2009-90035 provided with a restricting blade, a typical range of thickness of the restricting blade is from equal to or greater than 0.1 mm to equal to or less than several tens of mm in a direction of the object or the X-ray detecting device from the focal spot. In the X-ray imaging system which includes the movable diaphragm unit described above, a penumbra is unavoidably produced resulting from the “focal diameter” and the “thickness” of the restricting blade of the movable diaphragm unit in an outer periphery of the exposure range.

An object of the present invention is to provide an X-ray imaging system capable of reducing penumbra leaking out of a detector.

Hereinafter, in the invention in the present application, a point which corresponds to an electron beam flux beam spot and has a limited focal diameter on an electron beam incident surface of a target will be referred to as a “focal spot.”

Hereinafter, preferred embodiments of the present invention will be described in detail with reference to the attached drawings. The size, material, shape, relative arrangement and the like of the components described in the embodiments are not limiting the scope of the present invention.

### X-Ray Imaging System

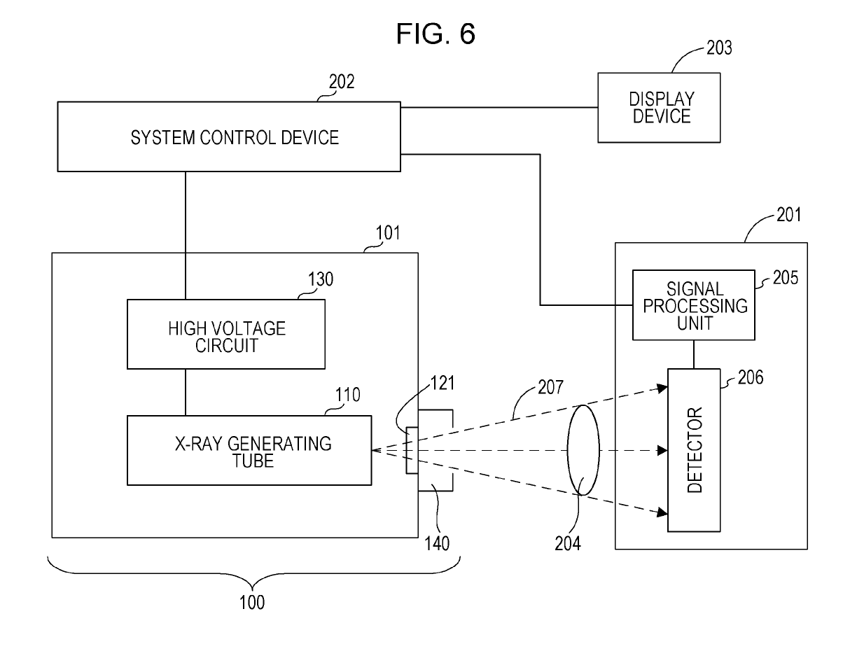

FIG. 6 is a configuration diagram of an X-ray imaging system of the present invention. In FIG. 6, a system control device 202 controls a radiation generating apparatus 100 and an X-ray detecting device 201 in a cooperated manner. A high voltage circuit 130 outputs various kinds of control signals to an X-ray generating tubes 110 under the control of the system control device 202. An emission state of X-ray emitted from the radiation generating apparatus 100 is controlled by the control signals. X-ray 207 emitted from an X-ray generating unit 101 is partially shielded by a movable diaphragm unit 140, passes through an object 204, and is detected by a detector 206. The detector 206 converts the detected X-ray into image signals and outputs the image signals to a signal processor 205. The signal processing unit 205 executes predetermined signal processing to the image signals under the control of the system control device 202 and outputs the processed image signals to the system control device 202. The system control device 202 outputs display signals to a display device 203 in order to display an image on the display device 203 in accordance with the processed image signals. The display device 203 displays an X-ray imaging image of the object 204 on a screen in accordance with the display signals.

### Radiation Generating Apparatus

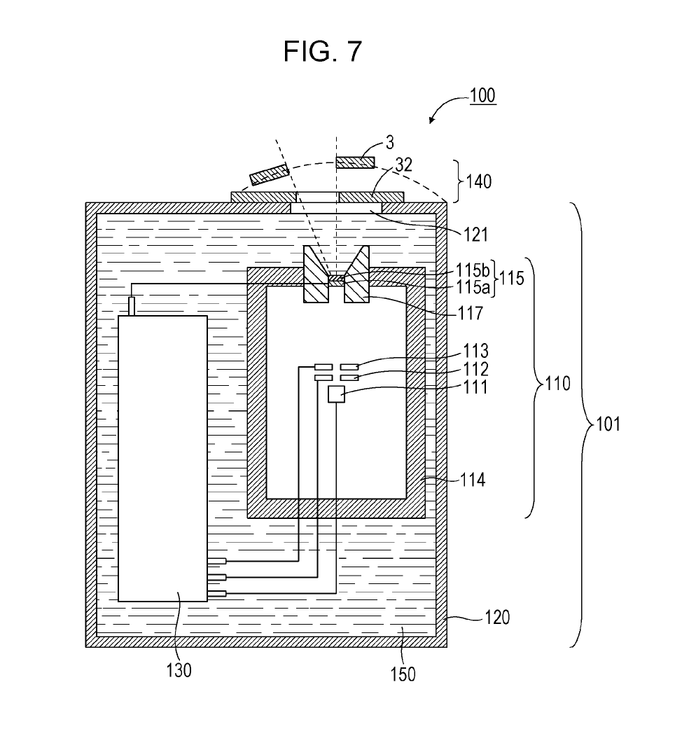

FIG. 7 is a diagram illustrating an exemplary configuration of the radiation generating apparatus 100 illustrated in FIG. 6.

The radiation generating apparatus 100 according to the present invention includes the X-ray generating unit 101 and the movable diaphragm unit 140. In the X-ray generating unit 101, the transmission X-ray generating tube 110 and the high voltage circuit 130 are housed in a container 102 that is filled with an insulating liquid 150. The insulating liquid 150 functions as an insulating medium and a cooling medium of the X-ray generating tube 110. It is desirable to use an electric insulating oil for the insulating liquid 150: specifically, mineral oil, silicone oil and the like are used suitably. Other examples of the insulating liquid 150 include a fluorine-based insulating liquid.

The high voltage circuit 130 generates a voltage which is applied to a cathode 111, a grid electrode 112, a lens electrode 113 and a target 115 of the X-ray generating tube 110. The target 115 consists of a target layer 115a fixed on a supporting substrate 115b which is made of a radiotransparent material. The cathode 111 is made of a tungsten filament, a hot cathode, such as an impregnated cathode, and a cold cathode. In a vacuum chamber 114, electrons are emitted toward the target 115 by an electric field formed by a grid electrode 112. The emitted electrons become an electron beam which is converged at the lens electrode 113, collides with the target layer 115a, and X-ray is emitted. At this time, unnecessary X-ray is shielded by an X-ray shielding member 117. The target layer 115a is made of, for example, tungsten, tantalum and molybdenum. After passing through the X-ray emission window 121, the X-ray is released outside under the limitation of an irradiation field by the movable diaphragm unit 140.

### X-Ray Imaging System

The X-ray imaging system of the present invention includes the radiation generating apparatus 100 and the X-ray detecting device 201 illustrated in FIG. 6, and has a feature in a configuration of the movable diaphragm unit 140 which is a component of the radiation generating apparatus 100.

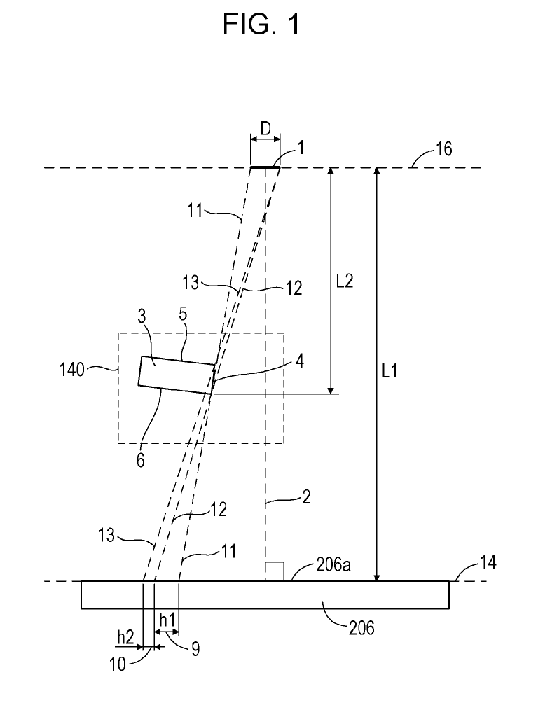

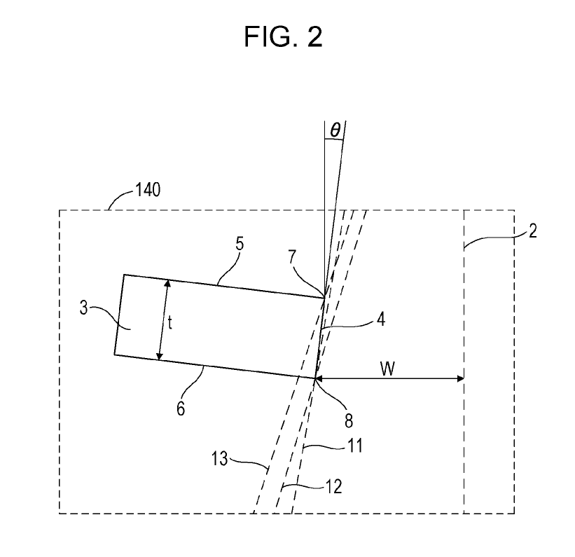

FIG. 1 is a schematic diagram illustrating the X-ray imaging system of the present invention. FIG. 1 illustrates the focal spot 1 of X-ray defined by the focal spot of the electron beam applied to the target layer 115a of the radiation generating apparatus 100 illustrated in FIG. 7, the restricting blade 3 of the movable diaphragm unit 140, and the detector 206 of the X-ray detecting device 201. FIG. 2 is an enlarged view of the movable diaphragm unit 140 of FIG. 1.

The movable diaphragm unit 140 according to the present invention includes the restricting blade 3 which restricts a passage range of X-ray, and a moving mechanism (not illustrated) of the restricting blade 3. As illustrated in FIG. 2, the restricting blade 3 has a predetermined thickness t defined by a front surface 5 on a target side and a back surface 6 of the opposite side of the front surface 5. The restricting blade 3 includes an end surface 4 which communicates with the front surface 5 and the back surface 6 on a side on which the X-ray passes. The moving mechanism (not illustrated) of the restricting blade 3 may move the restricting blade 3 in a direction in which the restricting blade 3 crosses an end surface 4.

The X-ray emitted from the focal spot 1 which has a limited size as described above passes through an opening formed by the restricting blade 3 (i.e., a right side area of the restricting blade 3 of FIG. 1) and forms an irradiation field on a detector plane 206a of the detector 206. At this time, an area is formed in a peripheral portion of the irradiation field in which a focal image is partially missed and an X-ray dose is low, i.e., an eclipsed penumbra 9. The eclipsed penumbra 9 is constituted by an area in which the restricting blade 3 covers the entire focal spot 1 and an area in which the restricting blade 3 does not cover the focal spot 1 when the focal spot 1 is seen from the detector plane 206a. That is, the eclipsed penumbra 9 is formed by the X-ray which is emitted from the focal spot 1, is in contact with the end surface 4, and then arrives at a first virtual plane 14 which includes the detector plane 206a. In FIG. 1, the eclipsed penumbra 9 is an area between a point at which X-ray 11 arrives at the first virtual plane 14 and a point at which X-ray 12 arrives at the first virtual plane 14. In FIG. 1, the X-ray 11 is emitted from an end portion of the focal spot 1 located nearer to the restricting blade 3, is in contact with a connection side 8 between the end surface 4 and the back surface 6 and arrives at the first virtual plane 14. The X-ray 12 is emitted from an end portion of the focal spot 1 located farther from the restricting blade 3, is in contact with the connection side 8 and arrives at the first virtual plane 14.

There is an area formed by X-ray partially passing therethrough because of a reduced effective thickness of the restricting blade 3 when seen from the irradiation side. This area is referred to as an attenuated penumbra 10. That is, the attenuated penumbra 10 is an area formed by the X-ray emitted from the focal spot 1, enters from the end surface 4 of the restricting blade 3, passes through the restricting blade 3, and arrives at the first virtual plane 14. In FIG. 1, the attenuated penumbra 10 is an area between a point at which X-ray 12 arrives at the first virtual plane 14 and a point at which X-ray 13 arrives at the first virtual plane 14. In FIG. 1, the X-ray 13 is emitted from an end portion of the focal spot 1 located farther from the restricting blade 3, is in contact with the connection side 7 between the front surface 5 and the end surface 4 of the restricting blade 3, and arrives at the first virtual plane 14.

The foregoing is defined by the positional relationships illustrated in FIGS. 1 and 2. Regarding the eclipsed penumbra 9 and the attenuated penumbra 10, positional relationships among the connection line 7, the connection line 8, the end surface 4, the front surface 5 and the back surface 6 may change depending on a posture of the restricting blade 3.

Since both the eclipsed penumbra 9 and the attenuated penumbra 10 are unnecessary components to the X-ray imaging image and may provide unnecessary exposure to the subject, it is desirable to reduce a width h1 of the eclipsed penumbra 9 and a width h2 of the attenuated penumbra 10. However, since the width of the eclipsed penumbra 9 is determined by the size of the focal spot 1 and an arrangement of the components, there is a limit to the reduction of the size of the eclipsed penumbra 9 from a viewpoint of the maintenance of predetermined X-ray intensity. In contrast, since the width of the attenuated penumbra 10 is determined by an angle of the end surface of the restricting blade 3, it is possible to reduce the attenuated penumbra 10 by the posture of the restricting blade 3.

In the present invention, in a case in which the width h1 of the eclipsed penumbra 9 and the width h2 of the attenuated penumbra 10 are not uniform at an arbitrary position of the restricting blade 3, h1 and h2 are defined by values obtained at a position nearest to a normal line 2 of the end surface 4.

Regarding the reduction of h2, according to the knowledge of the present inventors, if h1>h2, the X-ray of the attenuated penumbra 10 is attenuated when the X-ray partially passes through the restricting blade 3. Therefore, an effect of the exposure by the attenuated penumbra 10 is considered to be small enough.

Further, as a more quantitive threshold, a harmful effect caused by the unnecessary exposure is not increased significantly as long as the exposure dose is equal to or smaller than the exposure dose caused by, for example, natural X-ray. The exposure dose caused by natural X-ray is considered to be 2.4 mGy/year/person, that is, 6.6 μGy/day/person. An exposure dose of a chest X-ray is 0.15 mGy. If an unnecessary exposure dose by the chest X-ray does not exceed the exposure dose caused by natural X-ray, it is considered that attenuated penumbra by X-ray radiography has no effects.

In FIG. 1, a distance from the focal spot 1 to the detector plane 206a is denoted by L1, a distance from the focal spot 1 to the connection side 8 of the restricting blade 3 is denoted by L2, and a diameter of the focal spot 1 is denoted by D. As typical chest X-ray photographing conditions, when L1 is set to be 1000 mm, L2 is set to be 75 mm, D is set to be 1.5 mm, h1 is set to be 18.5 mm, and an irradiation area is set to be 300 mm×300 mm, an exposure area becomes 90000 mm². Regarding exposure by the eclipsed penumbra 9, when it is supposed that there is no attenuation caused by the thickness, an exposure ratio of the eclipsed penumbra 9 is 18.5×300×4/90000=0.247 with respect to the total exposure dose. Therefore, the exposed dose of the eclipsed penumbra 9 is 0.15×0.247×1000=37 μGy.

Generation of the eclipsed penumbra 9 is unavoidable but should be reduced. Here, an intensity ratio of the exposure dose caused by natural X-ray to the eclipsed penumbra 9 will be considered as an index of an intensity ratio of the attenuated penumbra 10 to the eclipsed penumbra 9. The intensity ratio of the exposure dose caused by natural X-ray with respect to the eclipsed penumbra 9 is 6.6/37=0.18. Therefore, when it is supposed that there is no reduction in dose after the X-ray passes through the restricting blade 3, if h2≦0.18×h1, the effect of the exposure of the attenuated penumbra 10 is considered to be equal to or smaller than that of the natural X-ray and can be tolerated.

As illustrated in FIG. 2, when a slope angle of the end surface 4 of the restricting blade 3 with respect to the normal line 2 is denoted by θ, a distance from the normal line 2 extending from the center of the focal spot 1 to the detector plane 206a to the connection side 8 is denoted by W, and a thickness of the restricting blade 3 is denoted by t, h1 and h2 are expressed by the following Expression:

$$h 1 = D ( \frac{L 1 - L 2}{L 2} )$$

$$h 2 = L 1 \vert \frac{W + \frac{D}{2} - t sin θ}{L 2 - t cos θ} - \frac{W + \frac{D}{2}}{L 2} \vert$$

### First Embodiment

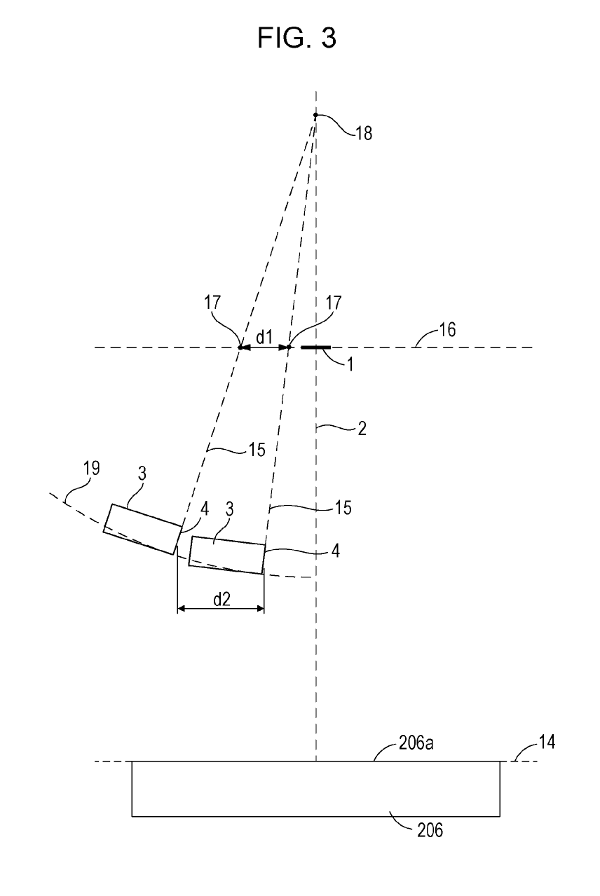

FIG. 3 is a cross-sectional view illustrating a specific moving form of the restricting blade 3 in an embodiment of the movable diaphragm unit 140 according to the present invention.

As a method for reducing the width h2 of the attenuated penumbra 10, a crossing line 17 at which a second virtual plane 15 in contact with the end surface 4 of the restricting blade 3 and a third virtual plane 16 which includes the focal spot 1 cross each other is first defined as illustrated in FIG. 3. Then, h2 may be reduced in the following manner: when the restricting blade 3 is moved away from the normal line 2, a moving distance d1 of the crossing line 17 with respect to the center of the focal spot 1 is set to be smaller than a moving distance d2 of the end surface 4 with respect to the normal line 2. As illustrated in FIG. 3, the moving distance d2 of the end surface 4 corresponds to a moving distance of the center of the end surface 4 in a direction parallel to the normal line 2.

For example, as illustrated in FIG. 3, a center of curvature 18 may be located on an extension of the normal line 2 on a front surface side of the restricting blade 3 and, on a curved surface which is convex toward the detector plane 206a, the restricting blade 3 may be moved so that the curved surface 19 and the end surface 4 maintain a predetermined angle. In this case, the second virtual plane 15 includes the end surface 4.

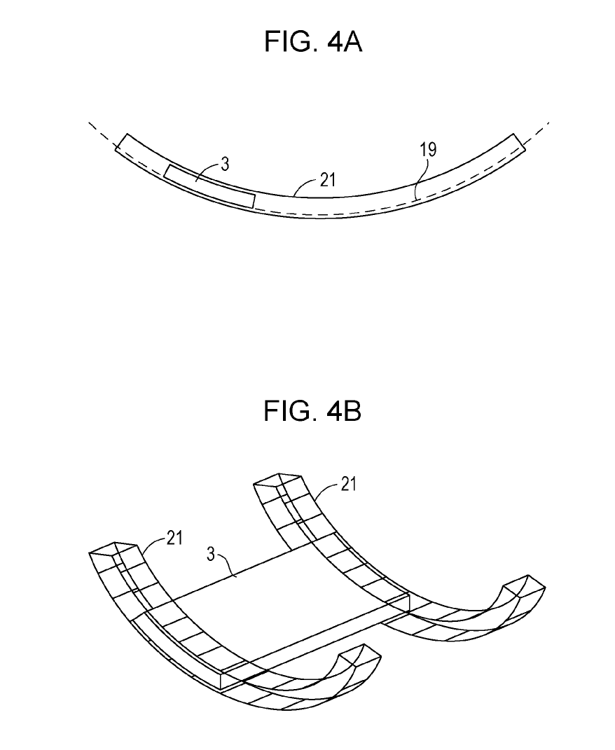

Exemplary moving mechanisms to implement the present embodiment will be described. As illustrated in FIG. 4A, a curved surface 19 of which center of curvature is located farther from the focal spot 1 with respect to the restricting blade 3 may be provided by providing a mechanism to cause the restricting blade 3 to slide along a guide groove 21 which forms the curved surface 19. As illustrated in FIG. 4B, guide grooves 21 may be provided on both sides of the restricting blade 3 and the restricting blade 3 may be operated using, for example, unillustrated slide tabs provided in the restricting blade 3. Alternatively, the restricting blade 3 may be desirably operated also by a mechanism using operation tabs and rack-pinion, a feed screw mechanism, a mechanism using a belt and a wire, and the like. Although the restricting blade 3 has a curved shape to conform the shape of the guide groove 21 in FIG. 4, a configuration in which projections are provided on side surfaces of a planar restricting blade and slidably inserted in the guide grooves 21 may also be employed.

According to this moving mechanism, since X-ray may be applied to the detector plane 206a while keeping the attenuated penumbra 10 small, it is desirable that the unnecessary exposure is made to the minimum. Further, since the restricting blade 3 is moved in a circular arc, a movement region of the restricting blade 3 may be made smaller than in a case in which the restricting blade 3 is moved in parallel, and the size of the restricting blade 3 may be reduced, it is desirable that the weight of the apparatus may be reduced.

### Second Embodiment

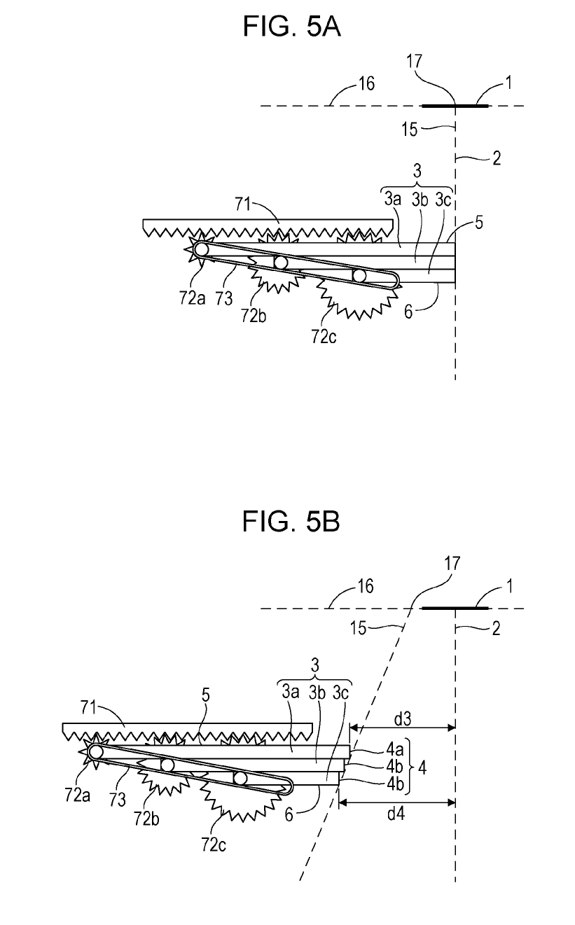

A specific moving form of a restricting blade 3 in the present embodiment is illustrated in FIGS. 5A and 5B. FIG. 5A illustrates a state in which an end surface 4 of the restricting blade 3 is in contact with a normal line 2 extending from the center of a focal spot 1 to a detector plane 206a. FIG. 5B illustrates a state in which the end surface 4 is spaced apart from the normal line 2.

In the present embodiment, the restricting blade 3 consists of a plurality of auxiliary blades 3a, 3b and 3c which are stacked in a thickness direction from a front surface 5 toward a back surface 6 so as to be relatively movable. As illustrated in FIG. 5B, when the restricting blade 3 is moved away from the normal line 2, the auxiliary blades 3a, 3b and 3c are moved in a shifted manner so that a moving distance d4 of the auxiliary blade 3c located on the back surface 6 side becomes longer than a moving distance d3 of the auxiliary blade 3a located on the front surface 5 side. The plurality of auxiliary blades 3a, 3b and 3c may have sliding surfaces between each blades as illustrated in FIGS. 5A and 5B.

In the present embodiment, the end surface 4 of the restricting blade 3 is divided into end surfaces 4a, 4b and 4c of the auxiliary blades 3a, 3b and 3c. Therefore, as illustrated in FIG. 5B, a second virtual plane in contact with the end surface 4 according to the present invention is defined as a plane in contact with each of end sides of the end surface 4a, 4b and 4c of the auxiliary blades 3a, 3b and 3c on back surface side.

Desirable examples of the moving mechanism of the auxiliary blades 3a, 3b and 3c include, as illustrated in FIGS. 5A and 5B, a rack-pinion gear mechanism provided with a rack 71, pinion gears 72a, 72b and 72c, a guide bar 73 and an unillustrated pulley. The rack 71 extends in directions in which three restricting blades 3a, 3b and 3c move. The three pinion gears 72a, 72b and 72c have engagement teeth which mesh with the rack 71. The number of the teeth increases in order of the pinion gears 72a, 72b and 72c. The pinion gears 72a, 72b and 72c rotatably connected to the auxiliary blades 3a, 3b and 3c, respectively. The guide bar 73 holds rotation axes of the three pinion gears 72a, 72b and 72c, and the unillustrated pulley aligns the number of rotations of the three pinion gears 72a, 72b and 72c.

According to the present embodiment, when the restricting blade 3 is to be moved, along a surface which crosses the normal line 2, the restricting blade 3 is moved while changing an angle made by a front surface 5 and the second virtual plane 15, and an amount of change in distance between a back surface 6 and the normal line 2 becomes longer than an amount of change in distance between a front surface 5 and the normal line 2.

The distance between the front surface 5 and the normal line 2 is defined by a distance between a side of the front surface 5 located nearer to the normal line and the normal line 2, and the distance between the back surface 6 and the normal line 2 is defined by a distance between a side of the back surface 6 located nearer to the normal line and the normal line 2.

### Third Embodiment

Applications of the first and the second embodiments will be described.

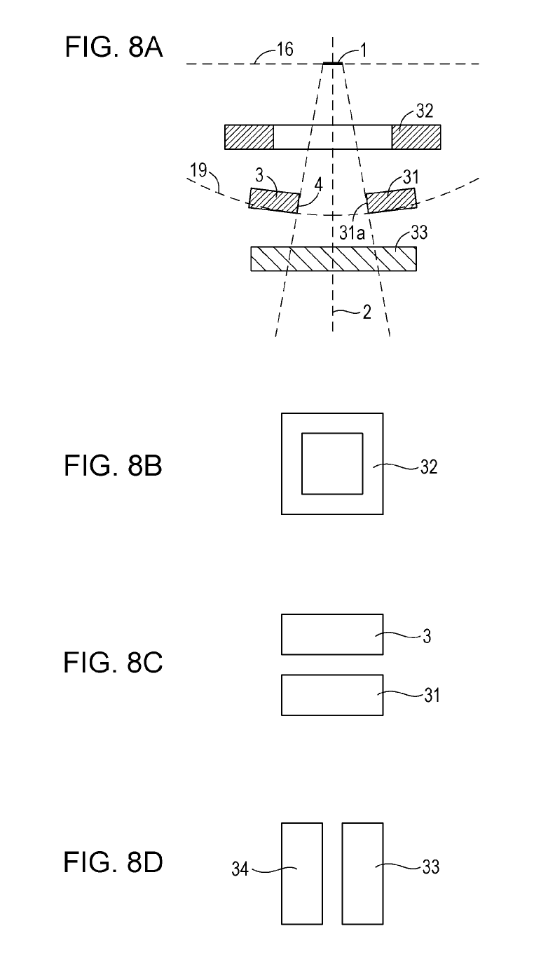

In the present invention, as illustrated in FIG. 8A, it is desirable that the restricting blade 3 illustrated in the first embodiment is used as a first restricting blade 3 and a second restricting blade 31 which has an end surface 31a facing the end surface 4 of the first restricting blade 3 is used. In the present embodiment, the first restricting blade 3 and the second restricting blade 31 are the same in shape. The first restricting blade 3 is moved by a first moving mechanism. The second restricting blade 31 is moved by a second moving mechanism which is substantially the same mechanism as that of the first restricting blade 3. Also in the second restricting blade 31, a width of an attenuated penumbra is defined to be smaller than a width of an eclipsed penumbra.

Further, in the present invention, as illustrated in FIG. 8A, a back blade 32 may be provided at a position further toward the focal spot 1 than the first restricting blade 3 and the second restricting blade 31. Alternatively, a combination of a third restricting blade 33 and a fourth restricting blade 34 may be disposed on a detector plane 206a side of the first restricting blade 3. Each of these restricting blades is illustrated in each of the plan views of FIGS. 8B to 8D.

FIG. 8B illustrates the back blade 32, FIG. 8C illustrates the first restricting blade 3 and the second restricting blade 31, and FIG. 8D illustrates the third restricting blade 33 and the fourth restricting blade 34. The third restricting blade 33 and the fourth restricting blade 34 are disposed so that moving directions thereof cross moving directions of the first restricting blade 3 and the second restricting blade 31. The third restricting blade 33 and the fourth restricting blade 34 are configured so that, as in the case of the first restricting blade 3, a width of an attenuated penumbra is smaller than a width of an eclipsed penumbra.

As illustrated in FIGS. 8A to 8D, an opening of arbitrary rectangular shape may be formed by using the second restricting blade 31, the third restricting blade 33 and the fourth restricting blade 34, and an X-ray field may be restricted to an arbitrary rectangular shape. Unnecessary X-ray may be shielded more effectively by using the back blade 32.

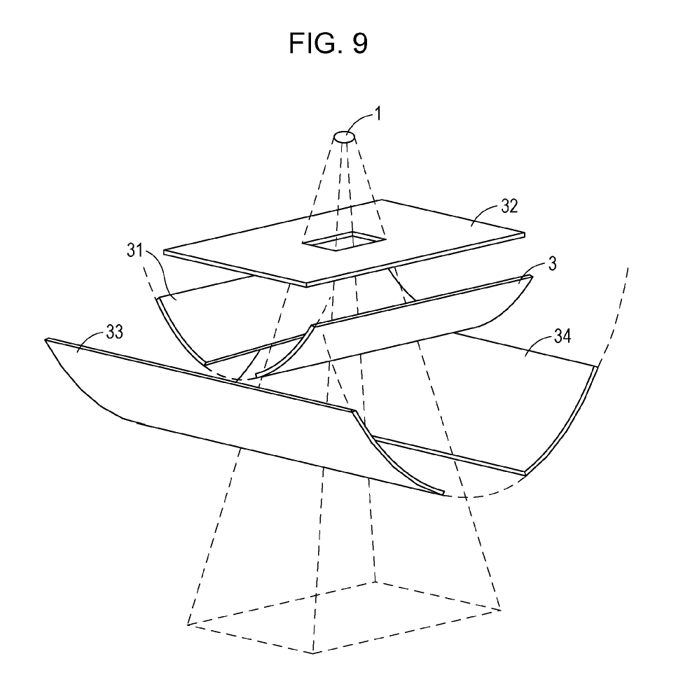

In the form in which the first restricting blade 3 to the fourth restricting blade 34 and the back blade 32 are combined as illustrated in FIGS. 8A to 8D, the first restricting blade 3 to the fourth restricting blade 34 may be formed in a curved surface shape as illustrated in FIG. 9. In the form described above, as illustrated in FIGS. 4A and 4B, the first restricting blade 3 to the fourth restricting blade 34 may be moved using guide grooves 21.

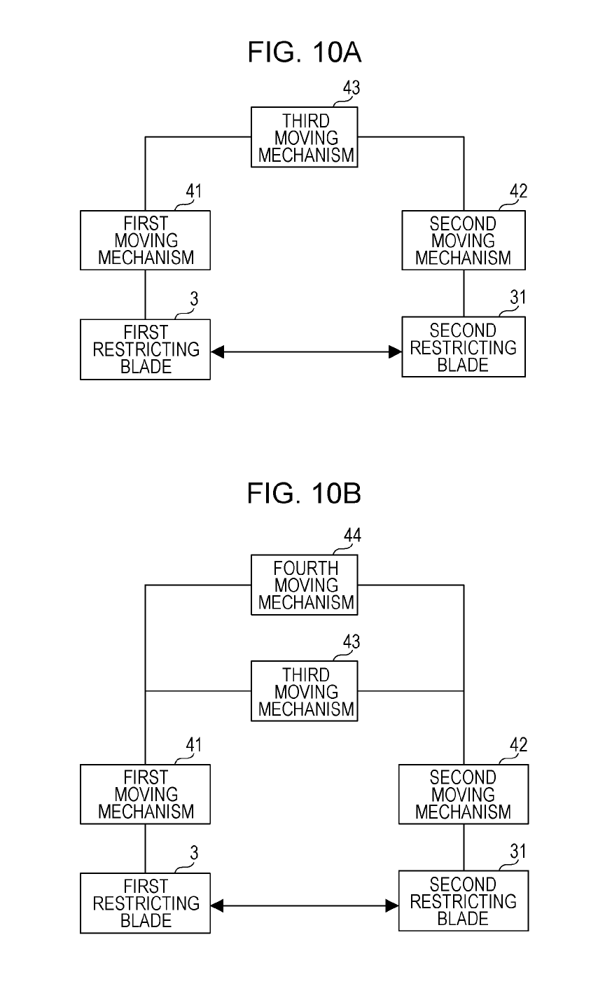

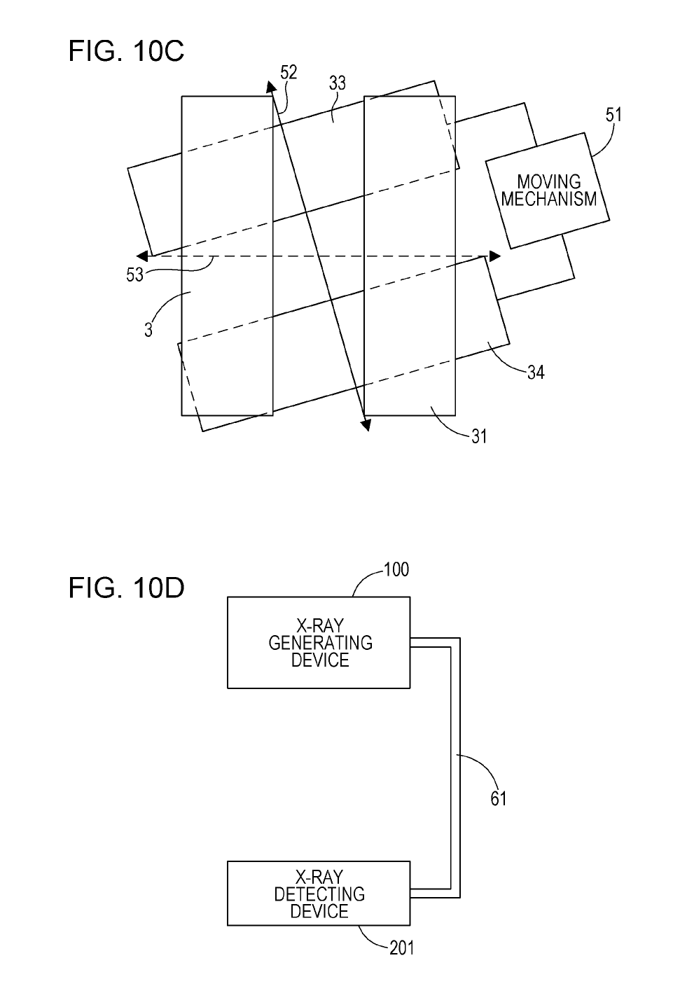

In the present invention, as illustrated in FIG. 10A, a third moving mechanism 43 connected to a first moving mechanism 41 which moves the first restricting blade 3 and to a second moving mechanism 42 which moves a second restricting blade 31 may be provided. A third moving mechanism 43 adjusts a distance between end surfaces 3 and 31 of these restricting blades by moving at least one of the first restricting blade 3 and the second restricting blade 31.

In the present invention, as illustrated in FIG. 10B, it is also desirable to provide a fourth moving mechanism 44 connected to the first moving mechanism 41 and to the second moving mechanism 42. The fourth moving mechanism 44 is a mechanism which moves the first restricting blade 3 and the second restricting blade 31 in an integrated manner while keeping a distance between the end surface 3 of the first restricting blade 3 and the end surface 31 of the second restricting blade 31.

In the present invention, in a form in which the third restricting blade 33 and the fourth restricting blade 34 illustrated in FIGS. 8A and 8B are provided, a fifth moving mechanism 51 which moves the third restricting blade 33 and the fourth restricting blade 34 in an integrated manner may be provided as illustrated in FIG. 10C. The fifth moving mechanism 51 moves the third restricting blade 33 and the fourth restricting blade 34 in a direction 53 in which the third restricting blade 33 and the fourth restricting blade 34 cross a moving direction 52 of the first restricting blade 3 and the second restricting blade 31.

Although applications of the first embodiment are described in the foregoing, the same applications may be made to the second embodiment.

In the present invention, as illustrated in FIG. 10D, a radiation generating apparatus 100 and an X-ray detecting device 201 may be connected by a support unit 61. In this configuration, a distance between a target of the radiation generating apparatus 100 and a detector plane 206a of the detector 206 of the X-ray detecting device 201 may be kept desirable.

### Example

A transmission radiation generating apparatus equipped with the movable diaphragm unit described in the first embodiment is configured and the X-ray imaging system of the present invention is configured. In the X-ray imaging system described above, a diameter D of the focal spot 1 is set to D=1.5 mm, a distance L from the focal spot 1 to the detector plane 206a is set to L1=1000 mm, a thickness t of the restricting blade 3 is set to t=2 mm, and a distance L between the focal spot 1 and the restricting blade 3 is set to L2=75 m. An angle θ made by the end surface 4 of the restricting blade 3 and the normal line 2 in a movable range of the restricting blade 3 when the opening becomes the largest is set to θ=15° and a distance W from the connection side 8 of the end surface 4 of the restricting blade 3 to the normal line 2 is set to W=20 mm.

A width h1 of the eclipsed penumbra 9 and a width h2 of the attenuated penumbra 10 are obtained as follows by the Expression above: h1=8.5 mm and h2=0.23 mm, which satisfy the relationship h2≦0.18×h1. Therefore, unnecessary exposure has been reduced sufficiently.

According to the present invention, unnecessary exposure caused by X-ray which has passed through a portion at which thickness of the restricting blade is insufficient when the opening of the restricting blade is adjusted can be reduced by devising arrangement of the restricting blade.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-232708, filed Nov. 11, 2013 which is hereby incorporated by reference herein in its entirety.

What is claimed is:

1. An X-ray imaging system, comprising: a radiation generating apparatus which includes an X-ray generating unit configured to emit X-ray from a target upon irradiation with an electron beam, and a movable diaphragm unit which includes a restricting blade configured to restrict a passage range of the X-ray and a moving mechanism of the restricting blade; and
an X-ray detecting device configured to detect, on a detector plane, X-ray emitted from the radiation generating apparatus and has passed through an object,
wherein the restricting blade has a predetermined thickness defined by a front surface on a target side and back surface of the opposite side of the front surface, and includes an end surface which communicates with the front surface and the back surface on a side on which the X-ray passes,
the moving mechanism moves the restricting blade in a direction in which the restricting blade crosses the end surface,
wherein, when a width of an eclipsed penumbra formed by X-ray emitted from a focal spot defined by an electron beam flux applied to the target, is in contact with the end surface, and arrives at a first virtual plane which includes the detector plane is denoted by h1 and
a width of an attenuated penumbra formed by X-ray emitted from the focal spot, enters from the end surface, passes through the restricting blade, and arrives at the first virtual plane is denoted by h2, a relationship h2<h1 is satisfied,
a second virtual plane which is in contact with the end surface crosses a third virtual plane which includes the focal spot,
when the restricting blade is moved away from a normal line extending from a center of the focal spot to the first virtual plane, a moving distance of a crossing line of the second virtual plane and a third virtual plane with respect to a center of the focal spot is shorter than a moving distance of the end surface with respect to the normal line.

2. The X-ray imaging system according to claim 1, wherein the relationship between h1 and h2 satisfies h2≦0.18×h1.

3. The X-ray imaging system according to claim 1, wherein, when the restricting blade is to be moved, a center of curvature is located on an extension of the normal line on the front surface side and, on a curved surface which is convex toward the detector plane from the focal spot, the restricting blade is moved while the curved surface and the end surface maintain a predetermined angle.

4. The X-ray imaging system according to claim 1, wherein, when the restricting blade is to be moved, along a surface which crosses the normal line, the restricting blade is moved while changing an angle made by the front surface and the second virtual plane, and an amount of change in distance between the back surface and the normal line is greater than an amount of change in distance between the front surface and the normal line.

5. The X-ray imaging system according to claim 4, wherein the restricting blade consists of a plurality of stacked auxiliary blades, the plurality of auxiliary blades being relatively movable in a direction perpendicular to a direction in which the auxiliary blades are stacked and, when the restricting blade is moved away from the normal line, a moving distance of an auxiliary blade on the back surface side is longer than a moving distance of the auxiliary blade on the front surface side.

6. The X-ray imaging system according to claim 5, wherein a moving mechanism of the restricting blade includes a rack-pinion gear mechanism.

7. The X-ray imaging system according to claim 1, wherein the movable diaphragm unit includes the restricting blade and the moving mechanism as a first restricting blade and a first moving mechanism and includes a second restricting blade which has an end surface facing an end surface of the first restricting blade and a second moving mechanism configured to move the second restricting blade.

8. The X-ray imaging system according to claim 7, wherein the movable diaphragm unit includes a third moving mechanism connected to the first moving mechanism and the second moving mechanism, and
the third moving mechanism adjusts a distance between an end surface of the first restricting blade and an end surface of the second restricting blade by moving at least one of the first restricting blade and the second restricting blade.

9. The X-ray imaging system according to claim 7, wherein the movable diaphragm unit includes a fourth moving mechanism connected to the first moving mechanism and the second moving mechanism, and
the fourth moving mechanism moves the first restricting blade and the second restricting blade in an integrated manner while keeping a distance between the end surface of the first restricting blade and the end surface of the second restricting blade.

10. The X-ray imaging system according to claim 7, wherein the movable diaphragm unit includes a third restricting blade and a fourth restricting blade of which end surfaces face each other, and a fifth moving mechanism which moves the third restricting blade and the fourth restricting blade in directions to cross moving directions of the first restricting blade and the second restricting blade, respectively.

11. The X-ray imaging system according to claim 1, further comprising a support unit which is connected to each of the X-ray detecting device and the radiation generating apparatus, disposed with a predetermined length in a direction to cross the detector plane, and supports the radiation generating apparatus at a predetermined height with respect to the detector.

12. An X-ray imaging system comprising: the X-ray imaging system according to claim 1; and a control device configured to control the radiation generating apparatus and the X-ray detecting device in the X-ray imaging system in a cooperated manner.

13. An X-ray generating apparatus, comprising: an X-ray generating unit configured to emit X-ray from a target upon irradiation with an electron beam, and a movable diaphragm unit which includes a restricting blade configured to restrict a passage range of the X-ray and a moving mechanism of the restricting blade; and
wherein the restricting blade has a predetermined thickness defined by a front surface on a target side and back surface of the opposite side of the front surface, and includes an end surface which communicates with the front surface and the back surface on a side on which the X-ray passes,
the moving mechanism moves the restricting blade in a direction in which the restricting blade crosses the end surface,
wherein the restricting blade consists of a plurality of stacked auxiliary blades, the plurality of auxiliary blades being relatively movable in a direction perpendicular to a direction in which the auxiliary blades are stacked and, when the restricting blade is moved away from the normal line, a moving distance of an auxiliary blade on the back surface side is longer than a moving distance of the auxiliary blade on the front surface side.

14. The X-ray generating apparatus according to claim 13, wherein, the X-ray emitted from a focal spot defined by an electron beam flux applied to the target,
when the restricting blade is to be moved, along a surface which crosses a normal line extending from a center of the focal spot, the restricting blade is moved while changing an angle made by the front surface and the second virtual plane, and an amount of change in distance between the back surface and the normal line is greater than an amount of change in distance between the front surface and the normal line.

15. The X-ray generating apparatus according to claim 13, wherein a moving mechanism of the restricting blade includes a rack-pinion gear mechanism.

16. A movable diaphragm unit device configured to be provided at forward respect to an extraction window of an X-ray generating unit generating an X-ray from a focal spot, comprising: a restricting blade configured to restrict a passage range of an X-ray and a moving mechanism of the restricting blade; and
wherein the restricting blade has an end surface on a side on which the X-ray passes,
the moving mechanism moves the restricting blade in a direction in which the restricting blade crosses the end surface,
wherein the restricting blade consists of a plurality of stacked auxiliary blades, the plurality of auxiliary blades being relatively movable in a direction perpendicular to a direction in which the auxiliary blades are stacked and, when the restricting blade is moved away from a normal line extending from a center of the focal spot, a moving distance of an auxiliary blade on the back surface side is longer than a moving distance of the auxiliary blade on the front surface side.
